(19) United States
(12) Patent Application Publication
(10) Pub. No.: US 2006/0184042 A1
(43) Pub. Date: Aug. 17, 2006

(54) Method, system and apparatus for dark-field reflection-mode photoacoustic tomography

(75) Inventors: Lihong Wang, College Station, TX (US); Konstantin Maslov, College Station, TX (US)

Correspondence Address: CHALKER FLORES, LLP, 2711 LBJ FRWY, Suite 1036, DALLAS, TX, 75234, US

(73) Assignee: The Texas A&M University System, College Station, TX (US)

(21) Appl. No.: 11/335,999

(22) Filed: Jan. 20, 2006

Related U.S. Application Data
(60) Provisional application No. 60/646,351, filed on Jan. 22, 2005.

(51) Int. Cl. A61B 6/00

(52) U.S. Cl. 600/476

(57) ABSTRACT

The present invention provides a method, system and apparatus for reflection-mode microscopic photoacoustic imaging using dark-field illumination that can be used to characterize a target within a tissue by focusing one or more laser pulses onto a surface of the tissue so as to penetrate the tissue and illuminate the target, receiving acoustic or pressure waves induced in the target by the one or more laser pulses using one or more ultrasonic transducers that are focused on the target and recording the received acoustic or pressure waves so that a characterization of the target can be obtained. The target characterization may include an image, a composition or a structure of the target. The one or more laser pulses are focused with an optical assembly of lenses and/or mirrors that expands and then converges the one or more laser pulses towards the focal point of the ultrasonic transducer.

### CROSS-REFERENCE TO RELATED APPLICATIONS

This application claims priority to U.S. Provisional Application Ser. No. 60/646,351, filed Jan. 22, 2005, the contents of which are incorporated by reference herein in their entireties.

### TECHNICAL FIELD OF THE INVENTION

The present invention relates generally to the fields of optics, lasers and medical diagnostic devices. More specifically, the present invention relates to a laser photoacoustic imaging system capable of producing a three-dimensional image (tomographic scan) of human organs.

### BACKGROUND OF THE INVENTION

The U.S. Government may own certain rights in this invention pursuant to the terms of the NIH Grant Nos. R01 EB000712 and R01 NS046214. Without limiting the scope of the invention, its background is described in connection with optics, lasers and medical photoacoustic imaging systems and diagnostic devices, as an example. The ability to image the micro-vascular network in skin is invaluable in dermatology and related cancer research. One of the promising techniques for accomplishing this objective is photoacoustic microscopy. A common goal of all imaging techniques is to achieve the highest possible resolution and contrast at the desired penetration depth within a reasonable time frame. Current high-resolution optical imaging techniques, such as confocal microscopy and optical coherence tomography, can image only up to approximately one transport mean free path (about 1 to 2 mm) into biological tissues because these techniques depend on ballistic or quasi-ballistic photons. These techniques are sensitive to the backscattering that is related to tissue morphology, but they are insensitive to the optical absorption that is related to important biochemical information. Photoacoustic imaging does not depend on ballistic or quasi-ballistic photons and can, therefore, penetrate deeper. Further, it provides high optical-absorption contrast while maintaining high ultrasonic resolution. Consequently, structures with high optical absorption coefficients, such as blood vessels, can be imaged clearly.

High image resolution has been achieved previously with circular-scanning photoacoustic computed tomography. Although a full 360-degree scan around an object provides high resolution and minimizes artifacts, it can be accomplished only on elevated objects, such as the brain or breast. The previously described planar reflection-mode techniques are not limited by the shape of the sample, but they may suffer from the strong photoacoustic waves emitted from optical absorbers near the surface (e.g., hair follicles or melanin) whose acoustic reverberations can potentially overshadow the much weaker photoacoustic signals from structures deep in the tissue.

Photoacoustic tomography (PAT), also referred to as optoacoustic tomography or thermoacoustic tomography, is a hybrid non-invasive imaging modality that combines high optical contrast with high ultrasonic resolution. As used herein, the terms photoacoustic, optoacoustic and thermoacoustic are synonymous. PAT uses a short laser pulse to excite ultrasonic waves in a medium and ultrasonic receiver(s) to detect the optical inhomogeneities-dependent ultrasonic waves to overcome the resolution disadvantages of optical imaging and the contrast and speckle artifact disadvantages of ultrasonography.

Laser photoacoustic imaging systems use time-resolved measurement of profiles of laser-induced transient pressure (acoustic) waves to enhance the axial resolution of the photoacoustic technique to a much finer value defined by the possibility of resolving two consecutive short pulses, i.e. to approximately lₐₓᵢₐₗ≈λ/3. This not only improves axial imaging resolution but also makes it possible to form 3D images of the media, at least within a layer of a thickness of the depth of field of the ultrasonic transducer. However, deep structures provide much smaller signals to the ultrasonic transducer than do near surface light-absorbing structures due to high optical attenuation and high ultrasonic absorption in biological tissues. In fact, high amplitude signals from near surface light-absorbing structures can completely obscure ultrasonic images of deeper structures due to acoustic scattering and reverberation even when the photoacoustic signals are time resolved.

Alternatively, optical contrast detection in turbid media is accomplished by heating of the absorbing media by laser pulses and using another optical beam of such a wavelength that it is not absorbed or scattered by the remainder of the medium for detection. The divergence of the beam is measured to indicate a change in the index of refraction of the medium due to absorption by the component. This technique excludes applications in a wide class of optically absorptive turbid media which includes, for example, practically all biological tissues.

Photoacoustic imaging devices for quantitative measurements, particularly the measurement of blood oxygenation, include a source of pulsed radiation and a probe with a front face that can be placed in close proximity to, or in contact with, a tissue site of an animal body. The probe further includes a plurality of optical fibers terminating at the surface of the front face of the probe and connecting at the other end to a pulsed laser. The front face of the probe also has mounted therein, or thereon, a transducer for detecting an acoustic response from blood in the tissue site through the radiation pulses connected to a processing unit which converts the transducer signals into a measure of blood oxygenation. The accuracy of these devices is limited by signals and changes in the amplitude of photoacoustic signals caused by local light absorbing structures (e.g., pigmented cells and blood vessels) located between the place where oxygenation is to be measured and the ultrasonic transducer.

Thermoacoustic or thermal wave microscopy has been used to detect surface and subsurface information from a material on a microscopic scale. A thermal wave is generated in a material by localized heating at a microscopic spot by focusing intensity modulated light, electromagnetic radiation, or a particle beam on the spot. The sample is scanned as a two-dimensional array of microscopic spots and the thermoacoustic signal that is produced in the surrounding gas is measured. This technique does not work in optically turbid media where sharp focusing of the light is impossible. In other words, with very few exceptions, it does not work in biological tissue.

Other systems image bulk acoustic emission sources induced by a single transient event of non-acoustic energy. For example, indirect photoacoustic emissions from cavitation-bubble formations where there is no coherence between the probing pulse and the emitted ultrasound. In this case, the depth localization must rely on the axial resolution of the focused ultrasonic transducer itself, which is reduced by the otherwise strong interference of the extraneous photoacoustic signals from the superficial paraxial.

A number of other systems do not use dark-field illumination. For example, laser ultrasound probes, suitable for intravascular use, which have an ultrasonic transducer and an optical fiber transmitting light from a laser source. Another system measures and characterizes the localized electromagnetic wave absorption properties of biologic tissues in vivo using incident pulses of electromagnetic waves and multiple acoustic transducers acoustically coupled to the surface of the tissue to measure the resultant acoustic waves. Yet another system uses a high aperture spherically focused transducer for the photoacoustic imaging of sub-surface tissue structures in medicine and biology. The technique uses a coaxial light pulse source transmitted through an optical fiber and a GRIN lens. Correspondingly, the peak intensity of the laser pulse is on the transducer axis near the tissue surface where it can produce high photoacoustic signals. A similar technique uses a ring piezoelectric transducer instead of a spherically focused one that allows an extended depth of field at the cost of worsened lateral resolution. Yet another technique uses several concentric rings and applies a dynamic focusing procedure that permits improved lateral resolution without sacrificing the imaging depth of field.

Accordingly there is a need for a method, system and apparatus for dark-field reflection-mode photoacoustic tomography that overcomes these disadvantages.

### SUMMARY OF THE INVENTION

The present invention provides a method, system and apparatus for reflection-mode microscopic photoacoustic imaging using dark-field illumination, as in dark-field microscopy. More specifically, the present invention uses a high-frequency large numerical-aperture (NA) spherically focused ultrasonic transducer that is coaxial and confocal with the optical illumination to achieve high image resolution and high sensitivity. By focusing light in such a way that it intersects a volume only within the depth of focus of the ultrasonic transducer, where a focused ultrasonic transducer is most sensitive to the ultrasonic waves emitted by high optical-contrast sources and has high resolution, the present invention significantly improves the imaging of relatively deep subsurface tissue structures which allows quantitative measurements to be performed in vivo by minimizing the interference caused by strong photoacoustic signals from superficial structures.

The present invention provides non-invasive optical-absorption imaging, including, but not limited to, imaging biological tissue in vivo up to a few centimeters deep where moderate resolution (e.g., on the order of tens to hundreds of microns) is sufficient. For example, the present invention is capable of imaging optical-absorption contrast in biological tissue up to 3 mm deep with a lateral resolution of about 45 to 120 micrometers. The broadband ultrasonic detection system provides high axial resolution, estimated to be about 15 micrometers. In addition, the present invention can be used to improve the photoacoustic monitoring of blood oxygenation by diminishing extraneous signals and changes in the amplitude of photoacoustic signals caused by local light absorbing structures (e.g., pigmented cells and blood vessels). Moreover, the present invention can utilize a variety of dyes to obtain aditional information.

As a result, the present invention has many advantages over existing bright-field illumination systems. First, the larger illumination area reduces the optical fluence on the sample surface so that more energy can be used while still conforming to ANSI safety standards (American National Standard for the Safe Use of Lasers, ANSI Standard Z136.D.H.). Secondly, the large illumination area partially averages out the shadows of superficial heterogeneity in the image. Thirdly, the dark-field illumination reduces the otherwise strong interference of extraneous photoacoustic signals from superficial paraxial areas.

The present invention utilizes the time-resolved detection of laser-induced ultrasonic waves to obtain three-dimensional images of the distribution of optical contrast within a sample volume. The main application of the technique is the imaging of internal organs or cellular structures of humans or animals for diagnostic or research purposes. The imaging procedure described herein, which uses a dark-field photoacoustic imaging system, is one of the possible embodiments, specifically the one aimed at medical and biological applications.

More specifically, the present invention provides a method of characterizing a target within a tissue by focusing one or more laser pulses onto a surface of the tissue so as to penetrate the tissue and illuminate the target, receiving acoustic or pressure waves induced in the target by the one or more laser pulses using one or more ultrasonic transducers that are focused on the target and recording the received acoustic or pressure waves so that a characterization of the target can be obtained. The target characterization may include an image of the target, a composition of the target or a structure of the target. This information can be used for diagnostic, treatment and research purposes. An image of the target can be displayed from the recorded acoustic or pressure waves. The recording of the received acoustic or pressure waves may also include digitizing the received acoustic or pressure waves and transferring the digitized acoustic or pressure waves to a computer for analysis. The one or more laser pulses are focused with a dark field condenser, which typically includes an optical assembly of lenses and/or mirrors (i.e., part of a photoacoustic sensor), that expands and then converges the one or more laser pulses towards the focal point of the ultrasonic transducer. The target can be a small volume of the tissue, such as an internal organ or cellular structure of a human or an animal.

In addition, the present invention provides various embodiments of a photoacoustic sensor that includes a focusing device and one or more ultrasonic transducers. The focusing device receives one or more laser pulses and focuses the one or more laser pulses onto a surface of a tissue so as to penetrate the tissue and illuminate the target. The one or more ultrasonic transducers are focused on the target and receive acoustic or pressure waves induced in the target by the one or more laser pulses. The photoacoustic sensor can be incorporated into a system that includes a pulsed laser and an electronic system that records and processes the received acoustic or pressure waves. The system can be used in a table top, portable, hand-held or catheter configuration.

The present invention can be used in the diagnostic screening of breast cancer (mammography), skin tumors and various other lesions (like port-wine stains etc.) whether accessible externally or via endoscopes, brain hematomas (hemorrhages), atherosclerotic lesions in blood vessels, and in the general characterization of tissue composition and structure. In addition, the present invention can provide quantitative information about tissue function, such as the level of blood oxygenation. With the help of tissue-specific dyes, the present invention can be used to identify tissue abnormalities such as superficial tumors. Finally, the present invention can be used to monitor possible tissue changes during drug, x-ray or other chemical or physical treatment or as a result of systematic application of cosmetics, skin creams, sun-blocks or other skin treatment products.

Additionally, by suppressing extraneous signals and diminishing the influence of optically absorptive (tissue) structures, such as melanomas and capillary vessels, on the amplitude of the photoacoustic signals, the present invention can dramatically improve quantitative measurements of tissue properties. That includes, for example, blood oxygenation monitoring, particularly in blood vessels.

### BRIEF DESCRIPTION OF THE DRAWINGS

The above and further advantages of the invention may be better understood by referring to the following description in conjunction with the accompanying drawings, in which:

FIG. 1 is a block diagram of a system that uses dark-field photoacoustic microscopy with contour scanning along the surface of the sample (following the profile of the surface) and quantitative spectroscopic measurement capability in accordance with the present invention;

FIG. 2. is a diagram of a photoacoustic sensor of the imaging system in accordance with one embodiment of the present invention;

FIG. 3 is a diagram of a photoacoustic sensor of the imaging system in accordance with another embodiment of the present invention;

FIG. 4 is a diagram of a photoacoustic sensor of the imaging system in accordance with yet another embodiment of the present invention;

FIG. 5 is a diagram of a photoacoustic sensor of the imaging system in accordance with yet another embodiment of the present invention;

FIG. 6 is a diagram of a photoacoustic sensor of the imaging system in accordance with yet another embodiment of the present invention;

FIG. 7 is a schematic diagram of a photoacoustic sensor of the imaging system in accordance with yet another embodiment of the present invention;

FIG. 8 is a schematic diagram of a photoacoustic sensor of the imaging system in accordance with yet another embodiment of the present invention;

FIG. 9 illustrates an image resolution test with a bar chart embedded 4 mm deep in a tissue phantom in accordance with the present invention;

FIG. 10 depicts an imaging depth test with a black double-stranded cotton thread embedded obliquely in the abdominal area of a rat in accordance with the present invention;

FIG. 11 depicts four in situ consecutive photoacoustic B-scans of the vascular distribution in rat skin in accordance with the present invention;

FIG. 12 depicts a comparison of the (a) in situ photoacoustic projection images taken from the epidermal side and (b) photographs taken from the dermal side with transmission illumination in accordance with the present invention; and

FIG. 13 is an in vivo non-invasive photoacoustic projection image taken from the epidermal side in accordance with the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a,” “an” and “the” are not intended to refer to only a singular entity, but include the general class of which a specific example may be used for illustration. The terminology herein is used to describe specific embodiments of the invention, but their usage does not delimit the invention, except as outlined in the claims.

To be consistent with the commonly used terminology, whenever possible, the terms used herein will follow the definitions recommended by the Optical Society of America (OCIS codes).

The term “photoacoustic microscopy” refers to a laser photoacoustic system that detects stress waves that are generated in the volume of a sample and passed to the irradiated tissue surface. In other words, photoacoustic microscopy is a procedure for obtaining images of the optical contrast of a material or tissue while detecting stress waves traveling from the object or the transient stress distribution within the volume of the sample.

The term “photoacoustic tomography” also refers to a laser photoacoustic system that detects stress waves that are generated in the volume of a sample and passed to the irradiated tissue surface, but the emphasis in this method is on computer-based image reconstruction.

As used herein, the term “piezoelectric detectors” refers to detectors of acoustic waves utilizing the principle of electric charge generation upon a change of volume within crystals subjected to a pressure wave.

As used herein, the terms “reflection mode” and “transmission mode” refer to a laser photoacoustic microscopy system that employs the detection of stress waves transmitted from the volume of their generation to the optically irradiated surface and a surface that is opposite to, or substantially different from, the irradiated surface, respectively.

As used herein, the term “transient stress waves” refers to stress waves that have a limited temporal duration and occupy a limited volume.

As used herein, the term “momentary stress” refers to a laser-induced stress within the sample volume during the course of laser energy deposition.

As used herein, the term “time-resolved detection” refers to the recording of the time history of transient stress waves with a temporal resolution sufficient to reconstruct a pressure wave profile.

As used herein, the terms “focused ultrasonic detector,” “focused ultrasonic transducer,” and “focused piezoelectric transducer” refer to an ultrasonic transducer of a hemispherical shape or a plane ultrasonic transducer attached to an acoustic lens, i.e., to an ultrasonic waveguide which has a hemispherical cavity on the other side. The transducer can be based on piezoelectric, capacitive, magnetostrictive, optical, or any other mechanisms.

As used herein, the term “transducer array” refers to an array of piezoelectric ultrasonic transducers.

The present invention provides a method, system and apparatus for reflection-mode microscopic photoacoustic imaging using dark-field illumination, as in dark-field microscopy. More specifically, the present invention uses a high-frequency large numerical-aperture (NA) spherically focused ultrasonic transducer that is coaxial and confocal with the optical illumination to achieve high image resolution and high sensitivity. By focusing light in such a way that it intersects a volume only within the depth of focus of the ultrasonic transducer, where a focused ultrasonic transducer is most sensitive to the ultrasonic waves emitted by high optical-contrast sources, the present invention significantly improves the imaging of relatively deep subsurface tissue structures which allows quantitative measurements to be performed in vivo by minimizing the interference caused by strong photoacoustic signals from superficial structures.

The present invention provides non-invasive optical-absorption imaging, including, but not limited to, imaging biological tissue in vivo up to a few centimeters deep where moderate resolution (e.g., on the order of tens to hundreds of microns) is sufficient. For example, the present invention is capable of imaging optical-absorption contrast in biological tissue up to about 3 mm deep with a lateral resolution of about 45 to about 120 micrometers. The broadband ultrasonic detection system provides high axial resolution, estimated to be about 15 micrometers. In addition, the present invention can be used to improve the photoacoustic monitoring of blood oxygenation by diminishing extraneous signals and changes in the amplitude of photoacoustic signals caused by local light absorbing structures (pigmented cells and blood vessels). Moreover, the present invention is complementary to the information about tissue structures that can be obtained from microwave-based techniques and it can utilize a variety of dyes to obtain additional information.

As a result, the present invention has many advantages over existing bright-field illumination systems. First, the larger illumination area reduces the optical fluence on the sample surface so that more energy can be used while still conforming to ANSI safety standards (American National Standard for the Safe Use of Lasers, ANSI Standard Z136.D.H.). Secondly, the large illumination area partially averages out the shadows of superficial heterogeneity in the image. Thirdly, the dark-field illumination reduces the otherwise strong interference of extraneous photoacoustic signals from superficial paraxial areas.

The present invention utilizes the time-resolved detection of laser-induced ultrasonic waves to obtain three-dimensional images of the distribution of optical contrast within a sample volume. The main application of the technique is the imaging of internal organs or cellular structures of humans or animals for diagnostic or research purposes. The imaging procedure described herein, which uses a dark-field photoacoustic imaging system, is one of the possible embodiments, specifically the one aimed at medical and biological applications.

As will be described below, the present invention provides a method of characterizing a target within a tissue by focusing one or more laser pulses onto a surface of the tissue so as to penetrate the tissue and illuminate the target, receiving acoustic or pressure waves induced in the target by the one or more laser pulses using one or more ultrasonic transducers that are focused on the target and recording the received acoustic or pressure waves so that a characterization of the target can be obtained. The target characterization may include an image of the target, a composition of the target or a structure of the target. This information can be used for diagnostic, treatment and research purposes. The recording the received acoustic pressure waves in the time domain can be performed by recording the received signal from the focused ultrasonic detector so that the characterization of the target in the axial direction can be obtained. An image of the target can be formed from the recorded acoustic or pressure waves. The recording and characterization of the target may also include digitizing the received acoustic or pressure waves and transferring the digitized acoustic or pressure waves to a computer for analysis. The one or more laser pulses are focused with a dark field condenser, which typically includes an optical assembly of lenses and/or mirrors (i.e., part of a photoacoustic sensor), that expands and then converges the one or more laser pulses towards the focal point of the ultrasonic transducer. The focused one or more laser pulses selectively heat the target where optical absorption is high causing the target to expand and produce a pressure wave whose temporal profile reflects the optical absorption and thermo-mechanical properties of the target. An annular array of transducers can be used along the tissue to enhance a depth of field of an imaging system by using a synthetic aperture image reconstruction. The target can be a small volume of the tissue, such as an internal organ or cellular structure of a human or an animal.

The present invention can be used in the diagnostic screening of breast cancer (mammography), skin tumors and various other lesions (like port-wine stains etc.) whether accessible externally or via endoscopes, brain hematomas (hemorrhages), atherosclerotic lesions in blood vessels, and in the general characterization of tissue composition and structure. In addition, the present invention can provide quantitative information about tissue function, such as the level of blood oxygenation. With the help of tissue-specific dyes, the present invention can be used to identify tissue abnormalities such as superficial tumors. Finally, the present invention can be used to monitor possible tissue changes during drug, x-ray or other chemical or physical treatment or as a result of systematic application of cosmetics, skin creams, sun-blocks or other skin treatment products.

The present invention provides an apparatus for imaging and characterizing a target within a tissue. The apparatus includes a focusing device and one or more ultrasonic transducers. The focusing device receives one or more laser pulses and focuses the one or more laser pulses onto a surface of a tissue to penetrate the tissue and illuminate the target. The one or more ultrasonic transducers are focused on the target and receive acoustic or pressure waves induced in the target by the one or more laser pulses.

The focusing device includes an optical assembly of one or more lenses and/or one or more mirrors that expand and then converge the one or more laser pulses toward the focal point of the one or more ultrasonic transducers. Furthermore, the one or more ultrasonic transducers are positioned coaxial and confocal with the one or more laser pulses. The one or more ultrasonic transducers that are focused on the target may be scanned in the form of an annular array of ultrasonic transducers along the tissue to enhance a depth of field of an imaging system by using a synthetic aperture image reconstruction. The focusing device may also include a dark field condenser.

In addition, the present invention may also include an electronic system in communication with the focusing device, the one or more ultrasonic transducers or a combination thereof. The electronic system includes an XYZ scanner, an amplifier, a digitizer, a computer, a processor, a display, a storage device or combination thereof. One or more component of the electronic system may be in communication remotely with the other components of the electronic system, the apparatus or both.

The present invention also provides a system for imaging and characterizing a target within a tissue. The system includes one or more pulsed lasers, a focusing device, one or more ultrasonic transducers and an electronic system. The focusing device is connected to an output of the one or more pulsed lasers that receives one or more laser pulses and focuses the one or more laser pulses onto a surface of a tissue so as to penetrate a tissue and illuminate a target. The one or more ultrasonic transducers are focused on the target and receive acoustic or pressure waves induced in the target by the one or more laser pulse. The electronic system records and processes the received acoustic or pressure waves. In addition, the electronic system may also include an XYZ scanner connected to the one or more ultrasonic transducers; an amplifier and/or digitizer connected to the one or more ultrasonic transducers; and a computer connected to the pulsed laser, the XYZ scanner, the amplifier, the digitizer or combinations thereof. The focusing device may include an optical assembly of one or more lenses and/or one or more mirrors that expand and then converge the one or more laser pulses toward the focal point of the one or more ultrasonic transducers.

Additionally, by suppressing extraneous signals and diminishing the influence of optically absorptive structures (e.g., tissue), such as melanomas and vessels, on the amplitude of the photoacoustic signals, the present invention can dramatically improve quantitative measurements of tissue properties. That includes, for example, blood oxygenation monitoring, particularly in blood vessels.

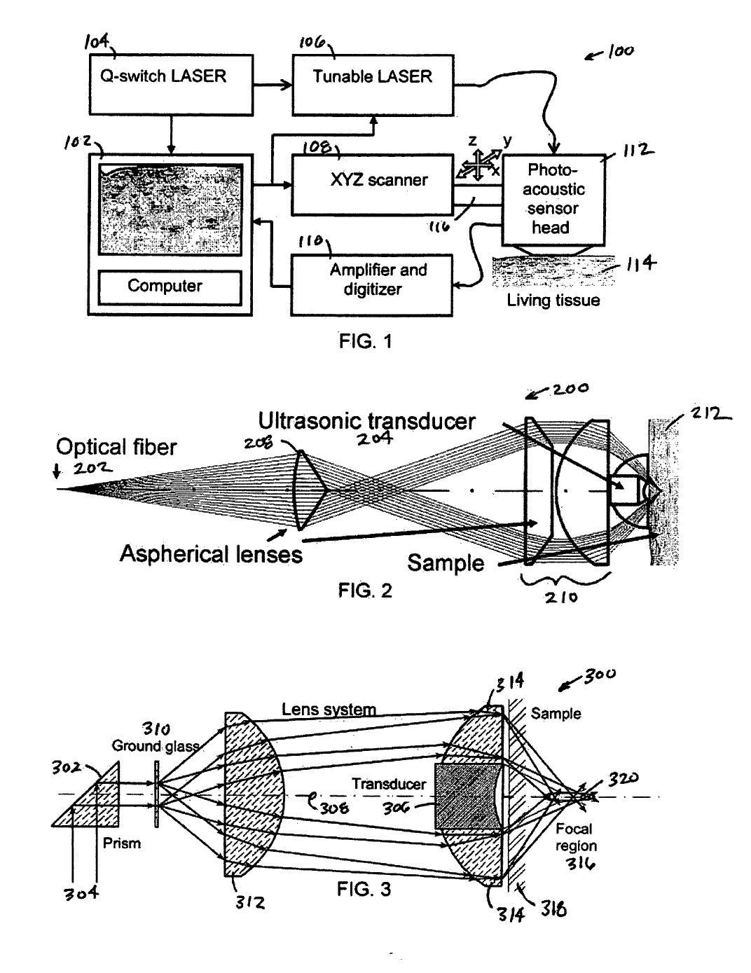

Now referring to FIG. 1, a block diagram of a system 100 that uses a dark-field photoacoustic microscopy with contour scanning along the surface of the sample (e.g.,following the profile of the surface) and quantitative spectroscopic measurement capability in accordance with the present invention is shown. The system 100 includes a pulsed laser (Q-switch laser 104 and tunable laser 106), a focusing device 112, one or more ultrasonic transducers 112 and an electronic system (computer 102, XYZ scanner 108 and amplifier and digitizer 110). The focusing device 112 is connected to an output of the pulsed laser 106 that receives one or more laser pulses and focuses the one or more laser pulses onto a surface of a tissue 114 so as to penetrate the tissue and illuminate a target. The one or more ultrasonic transducers 112 are focused on the target and receive acoustic or pressure waves induced in the target by the one or more laser pulses. The electronic system (102, 108 and 110) records and processes the received acoustic or pressure waves. As will be described below, the focusing device includes an optical assembly of lenses and/or mirrors that expand and then converge the one or more laser pulses toward the focal point of the one or more ultrasonic transducers.

The dark field confocal photoacoustic sensor 112 is placed on a motorized platform 116 to perform raster scanning along the tissue surface 114 with simultaneous adjustments of the sensor axial position to compensate for the curvature of the tissue surface. The recorded pressure-wave time histories are displayed by the computer 102 versus the photoacoustic sensor 112 position to construct three dimensional images of the distribution of the optical contrast within the tissue 114 which produces a three dimensional tomographic image of the tissue structure.

To obtain functional images, laser pulses from a tunable laser 106 (such as, e.g., a dye laser) are used to illuminate the tissue surface. By switching between several light wavelengths, the optical absorption spectrum of a tissue structure can be measured. This spectrum is influenced by the dispersion of optical absorption and scattering in the surrounding media. Nevertheless, in cases where the tissue absorption has definite and distinct spectral features, which is the case for the example with oxyhemoglobin and deoxyhemoglobin, by using a proper minimization procedure, it is possible to separate the contributions of different tissue constituents, and thus permit the measurement of local blood oxygenation in the tissue in order to separate normal and diseased tissues. Similarly, certain tumors can be identified by targeting them with biomolecules conjugated to various contrast agents such as selectively absorbing dyes.

The one or more short laser pulses are delivered to the front surface of a sample (e.g., human or animal body, tissue or organ) under investigation, thus illuminating the sample outside the ultrasonic transducer aperture. The laser wavelength is selected as a compromise of desirable light penetration depth and high contrast between the structures of interest and the surrounding media. Light absorption by the internal structures causes a transient temperature rise which, due to thermoelastic expansion of the media, produces elastic waves that can travel outside the sample volume.

In media with strong light scattering, such as biological tissue, it is impossible to achieve sharp focusing of the optical beam beyond one optical transport mean free path, and, correspondingly, the photoacoustic image resolution is primarily determined by the ultrasonic detection parameters. Short laser pulses are needed to confine the thermo-elastic expansion to the volume of high optical absorption, that is, to diminish the effects of thermal diffusion during the laser pulse, and to generate high frequency elastic waves in order to obtain images with high spatial resolution.

However, increasing the ultrasonic frequency too much can also result in an undesirably small penetration depth because the strong frequency dependence of ultrasonic attenuation in turbid media, e.g., in biological tissue about 0.7 to about 3 dB/(cm MHz) increases linearly with the frequency. Therefore, the photoacoustic signal from optical contrast at the maximum penetration depth might have an amplitude much smaller than that from the superficial area because of the smaller magnitudes of the optical pulse reaching the place of interest and the ultrasonic waves returning to the ultrasonic transducer. Ultrasonic pulses from superficial sources propagating in a complex environment produce reverberations due to multiple reflections from the sample's heterogeneities, the sample and ultrasonic transducer surfaces, and the boundaries of the elements of the ultrasonic transducer. Reverberations of a very strong ultrasonic pulse produce a quasi-random signal of amplitude comparable to that of a photoacoustic signal from deep subsurface optical contrast structures. This effect sharply decreases the signal-to-noise ratio of the photoacoustic signal and, consequently, the quality of the photoacoustic image and frequently makes quantitative measurements based on photoacoustic imaging difficult.

The present invention overcomes the above mentioned problem by expanding the laser light beam, passing it outside the lens aperture and focusing it through an optical condenser in such a manner that the optical focal region overlaps with the focal spot of the ultrasonic transducer, thus forming a confocal optical dark-field illumination within the depth of field of ultrasonic transducer.

High-frequency ultrasonic waves generated in tissue by the laser pulse are recorded and analyzed by a computer 102 to reconstruct a three-dimensional image. The shape and dimensions of the optical-contrast tissue structures are generally determined from the temporal profile of the laser-induced ultrasonic waves and the position of the focused ultrasonic transducer 112. Ordinarily, a raster scan by the ultrasonic transducer is used to form a three-dimensional image. However, a transducer array can be used to reduce the time of scanning and the incident light fluence. When the tissue under investigation is an internal organ, the optical fiber and transducer may be incorporated in an endoscope and positioned inside the body. The following examples are provided for the purpose of illustrating various embodiments of the invention and are not meant to limit the present invention in any fashion.

The present invention includes any realization of dark-field light condensers using any kind of mirrors, lenses and aperture diaphragms which can produce illumination confined to the focal area of the focused ultrasonic transducer or produce less intense light outside the imaging depth of field of the ultrasonic transducer or transducers. The following devices can implement the method described herein: an optical condenser of a different design in which lenses close to the transducer were replaced with conical mirrors; a conical lens was replaced with an ordinary spherical lens; a system of prisms or mirrors was used for light delivery instead of optical fibers; contour scanning along the surface of the sample (following the profile of the surface) was performed instead of raster x-y scanning. Various examples of photoacoustic sensors will now be described in reference to FIGS. 2-8 wherein the photoacoustic sensor includes a focusing device and one or more ultrasonic transducers. The focusing device receives one or more laser pulses and focuses the one or more laser pulses onto a surface of a tissue so as to penetrate the tissue and illuminate the target. The one or more ultrasonic transducers are focused on the target and receive acoustic or pressure waves induced in the target by the one or more laser pulses.

Now referring to FIG. 2, a diagram of a photoacoustic sensor 200 of the imaging system in accordance with one embodiment of the present invention is shown. A Q-switched pulsed Nd:YAG laser (e.g., Brilliant B, BigSky), operating at about 532 nm, delivers about 300-μJ per pulse to about a 0.60 -mm diameter optical fiber 202. The laser pulse width is about 6.5 ns, and the pulse repetition rate is about 10 Hz. The fiber output 202 is coaxially positioned on a three-dimensional precision mechanical scanner with a focused ultrasonic transducer 204 (e.g., Panametrics). The transducer 204 has a center frequency of about 50 MHz and a nominal bandwidth of about 70% and is attached to a concave lens 206 (e.g., aperture diameter (D) of about 5.5 mm and focal length (F) of about 5.6 mm). This aperture provides an NA of about 0.44, which is considered relatively large in ultrasonics. The laser light from the fiber 202 is expanded by a conical lens 208 and then focused through an optical condenser 210 with an NA of about 1.1. The optical focal region overlaps with the focal spot of the ultrasonic transducer 204, thus forming a confocal optical dark-field illumination and ultrasonic detection configuration.

Photoacoustic signals received by the ultrasonic transducer 204 are amplified by a low-noise amplifier (ZFL-500LN, Mini-Circuits) and recorded by a digital oscilloscope. The transducer 204 is immersed in water inside a plastic container with an opening at the bottom that is sealed with a thin disposable polyethylene membrane. The sample 212 (animal) is placed outside the container below the membrane, and the ultrasonic coupling is further secured by coupling gel. The skilled artisan will recognize that the individual components of the present invention may be constructed in part or entirely out of a variety of materials, e.g., plastics, polymers, metals alloys, rubbers, composites, glasses, crystals, ground glass, quartz, and so forth.

Compared to alternative designs involving bright-field illumination, the above design provides the following advantages. First, the large illumination area reduces the optical fluence on the sample surface to less than 1 mJ/cm², which is well within the safety standards. Secondly, the large illumination area partially averages out the shadows of superficial optical absorbers in the image. Thirdly, the dark-field illumination reduces the otherwise strong interference of extraneous photoacoustic signals from superficial paraxial areas.

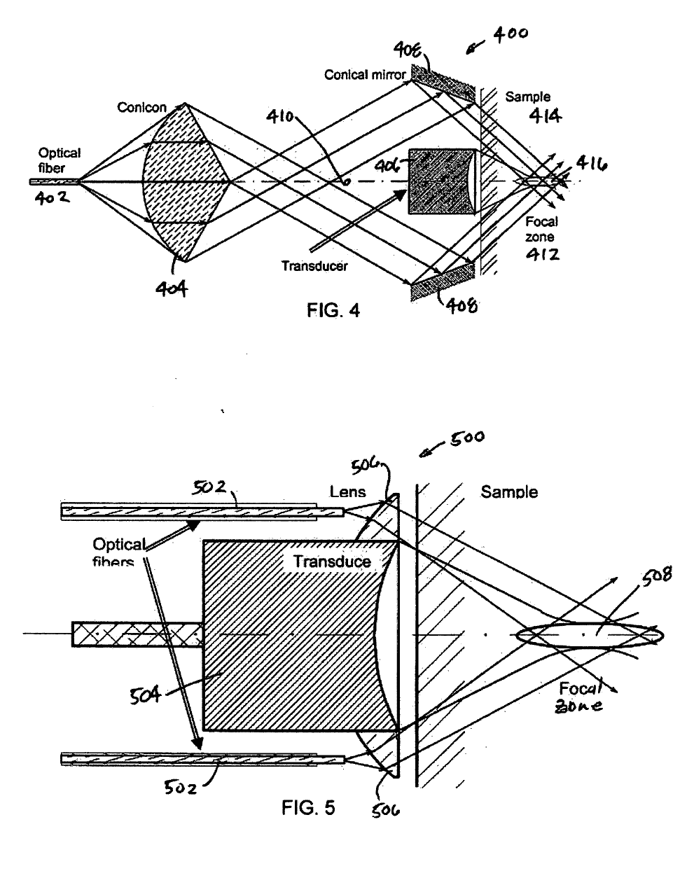

Referring now to FIG. 3, a diagram of a photoacoustic sensor 300 of the imaging system in accordance with another embodiment of the present invention is shown. The dark-field confocal photoacoustic sensor 300 uses a set of prisms 302 to focus the light pulse 304 onto the focal plane of the ultrasonic transducer 306. In this particular realization, the laser pulse 304 is delivered via a set of right angle prisms 302, directed along the ultrasonic transducer axis 308, diffused by a narrow angle diffuser 310 (e.g., ground glass), expanded using a conical lens 312, passed around the ultrasonic transducer 306, and focused by a set of annular plano-convex lenses 314. The laser pulse along the ultrasonic transducer axis 308 is confined to the transducer's depth of focus 316. The laser pulse penetrates through the surface of the tissue 318 to a sufficient depth, selectively heating a volume of the tissue 320 with higher optical absorption and producing ultrasonic waves which propagate with minimal alteration to the tissue surface. The ultrasonic waves are detected by an acoustic transducer 306, digitized and transferred to a computer for data analysis.

Now referring to FIG. 4, a diagram of a photoacoustic sensor 400 of the imaging system in accordance with yet another embodiment of the present invention is shown. A laser pulse is delivered via optical fiber 402, expanded by a conical len 404, passed around the ultrasonic transducer 406, and focused by a conical mirrors 408. The laser pulse along the ultrasonic transducer axis 410 is confined to the transducer's depth of focus 412. The laser pulse penetrates through the surface of the tissue 414 to a sufficient depth, selectively heating a volume target 416 of the tissue 414 with higher optical absorption and producing ultrasonic waves which propagate with minimal alteration to the tissue surface. The ultrasonic waves are detected by an acoustic transducer 406, digitized and transferred to a computer for data analysis.

Referring now to FIG. 5, a diagram of a photoacoustic sensor 500 of the imaging system in accordance with yet another embodiment of the present invention is shown. Photoacoustic sensor 500 can be integrated into a one- or two-dimensional portable scanner 500 (e.g., in some instances the system may be a hand-held scanner) in place of a 3D stationary scanning table 100, e.g., FIG. 1. Other possibilities include the use of multiple optical fibers 502 positioned around an ultrasonic transducer 504 to increase the optical fluence and miniaturize the photoacoustic sensor 500. The transducer 504 is attached to a concave lens 506. The laser light from the optical fibers 502 is focused through an optical condenser 506. The optical focal region overlaps with the focal spot 508 of the ultrasonic transducer 504, thus forming a confocal optical dark-field illumination and ultrasonic detection configuration.

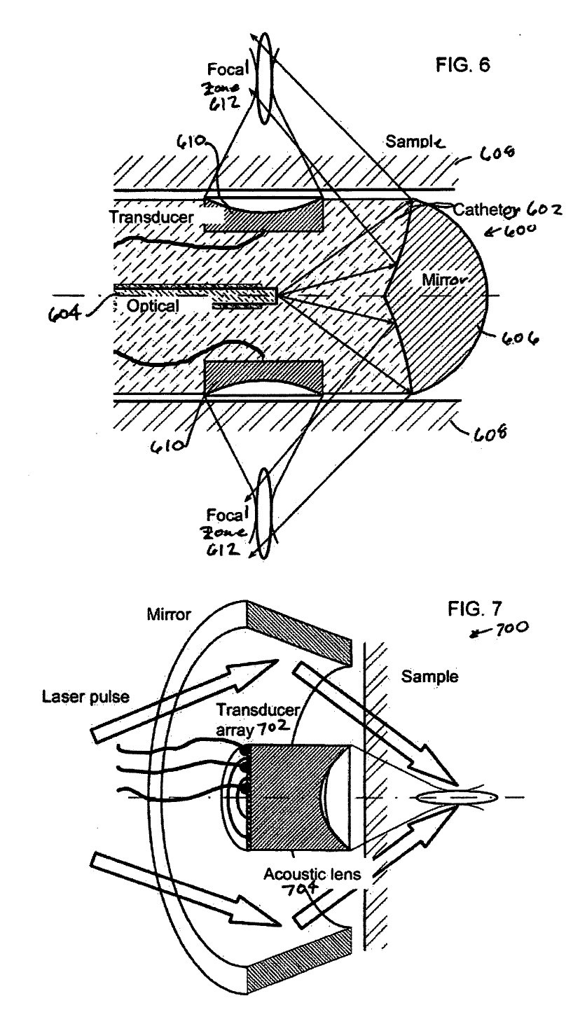

Now referring to FIG. 6, a diagram of a photoacoustic sensor 600 of the imaging system in accordance with yet another embodiment of the present invention is shown. A multiple element piezoelectric transducer can be used as a photoacoustic sensor 600, which is one of the possible embodiments suitable for intravascular imaging. Although, this embodiment is discussed in terms of intravascular, the skilled artisan will recognize that the invention may be used in any region that will accept a catheter or tube. Such a device can further improve photoacoustic imaging by accelerating the image collection time due to the simultaneous recording of photoacoustic signals from different points and the miniaturizing of the photoacoustic sensor 600 by eliminating the mechanical scanning apparatus. The photoacoustic sensor 600 is integrated into a catheter 602 that includes a coaxial mounted fiber optic cable 604 and a mirror 606 disposed towards the end of the catheter 602. A protective end (not shown) can be used to protect the photoacoustic sensor 600 and improve entry and manipulation within the sample 608. The catheter 602 also includes one or more transducers 610 that are positioned so that the optical focal region from mirror 606 overlaps with the focal spot 612 of the ultrasonic transducer(s) 610, thus forming a confocal optical dark-field illumination and ultrasonic detection configuration.

Referring now to FIG. 7, a diagram of a photoacoustic sensor 700 of the imaging system in accordance with yet another embodiment of the present invention is shown. Photosensor 700 is similar to photosensors 200 (FIG. 2), 300 (FIG. 3), 400 (FIG. 4) and 500 (FIG. 5), except that the single-element focused ultrasonic transducer is replaced with a multi-element annular piezoelectric transducer array 702 coupled to an ultrasonic lens 704. The ultrasonic transducer 702 can be dynamically focused to different depths for a single laser pulse by introducing time-of-flight-dependent time delays between signals from different transducer elements, thus extending the area of the cross-sectional (B-scan) image with high lateral resolution.

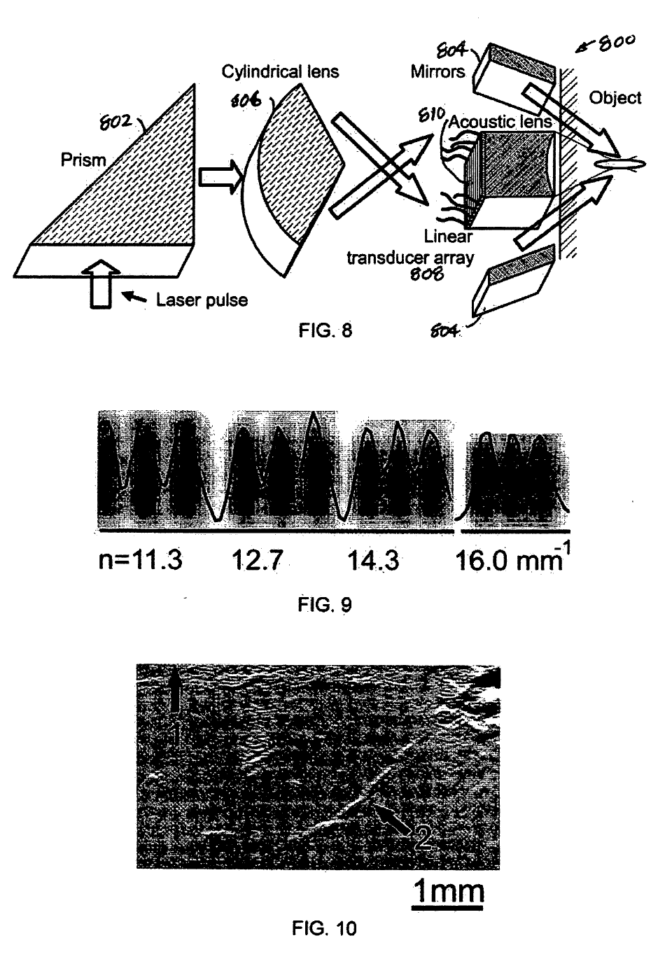

Now referring to FIG. 8, a diagram of a photoacoustic sensor 800 of the imaging system in accordance with yet another embodiment of the present invention is shown. Photoacoustic sensor 800 uses a system of prisms 802, mirrors 804 and cylindrical lenses 806 to deliver light pulses, and a one dimensional cylindrically focused transducer array 808 and acoustic lens 810 to form a photoacoustic B-scan image. In this embodiment, the photoacoustic sensor 800 uses translational symmetry instead of cylindrical symmetry. Unlike the embodiments shown in FIGS. 2, 3, 4 and 5, a wedge-shaped light beam is formed instead of a cone-shaped one, and a linear transducer array 808 coupled with a cylindrical ultrasonic lens 810, similar to one used in medical ultrasonic diagnostics, is used to acquire photoacoustic signals. Using dynamic focusing in the direction along the ultrasonic axis, such a device can produce a complete photoacoustic B-scan image with a single laser pulse to make possible real time photoacoustic imaging. Finally, the single row of piezoelectric elements (one-dimensional array) can be replaced by several parallel rows of elements (two-dimensional array) for three-dimensional imaging.

Referring now to FIG. 9, an image resolution test with a bar chart embedded 4 mm deep in a tissue phantom in accordance with the present invention is shown. The four photoacoustic images of a Mylar USAF-1951 target taken through a 4 -mm thick layer of light diffusing tissue phantom made from about 2% Intralipid solution (Clintec Nutrition Co., Dearfield, Ill.) and about 1% agar gel. The estimated reduced scattering coefficient of the phantom, μ′ₛ≈1.5 mm⁻¹, is greater than that of most biological tissues. The thickness of the phantom translates into about six transport mean free paths. The numbers below the images indicate the spatial modulation frequency (v), expressed in line-pairs/mm. The solid curves show the relative ultrasonic pressure as a function of the horizontal displacement across the bars on the target. The modulation transfer function was extracted and extrapolated to its cut-off spatial frequency, producing an estimated lateral resolution of about 45 μm. On the other hand, the axial resolution was estimated to be about 15 μm based on the spread function of the photoacoustic signal from the top surface of an embedded object.

When a 1.2 -mm thick freshly harvested skin from a sacrificed rat was placed between the acoustic lens and the USAF-1951 target, the lateral resolution degraded to about 120 μm, which was likely due to increased ultrasonic attenuation. Ultrasonic attenuation in the skin decreased the signal-to-noise ratio (SNR) to about 30 dB from about 80 dB in clear water and about 50 dB in the phantom samples. Increasing the SNR may potentially recover the resolution.

Now referring to FIG. 10, an imaging depth test with a black double-stranded cotton thread embedded obliquely in the abdominal area of a rat in accordance with the present invention is shown. A photoacoustic B-scan (vertical cross section) image of a black double-stranded cotton thread of about 0.2 mm in diameter and about 1¼ mm in pitch, which was embedded obliquely in the abdominal area of a sacrificed rat. The image illustrates the surface of the skin indicated by arrow 1 and the black double-stranded cotton thread embedded obliquely in the abdominal area of a rat by arrow 2. The thread is clearly visible in the image up to 3 mm in depth, which shows the capability of one embodiment of the present invention.

Referring now to FIG. 1 1, four in situ consecutive photoacoustic B-scans of the vascular distribution in rat skin in accordance with the present invention are shown. The photoacoustic images show the vascular distribution in the dorsal dermis (e.g., the upper lumbar area to the left of the vertebra) of a Sprague Dawley rat (e.g., about 180 g, Charles River Breeding Laboratories). Before imaging, the hair on the back of the rats was removed using commercial hair remover lotion. Imaging was performed under general anesthesia by intramuscular injection of ketamine hydrochloride (about 44 mg/kg), xylazine hydrochloride (about 2.5 mg/kg), acepromazine maleate (about 0.75 mg/kg), and atropine (about 0.025 mg/kg). During the procedure, the animals' normal body temperature was maintained by controlling the immersion container, with additional heat provided as needed by an overhead surgical lamp. After several hours of experimentation, the rats recovered normally without noticeable health problems. Finally, the rats were sacrificed using pentobarbital (e.g., about 120 mg/kg, IP). The imaged skin was removed from the rats and photographed from the inner skin surface. All experimental animal procedures were carried out in compliance with the guidelines of the United States National Institutes of Health.

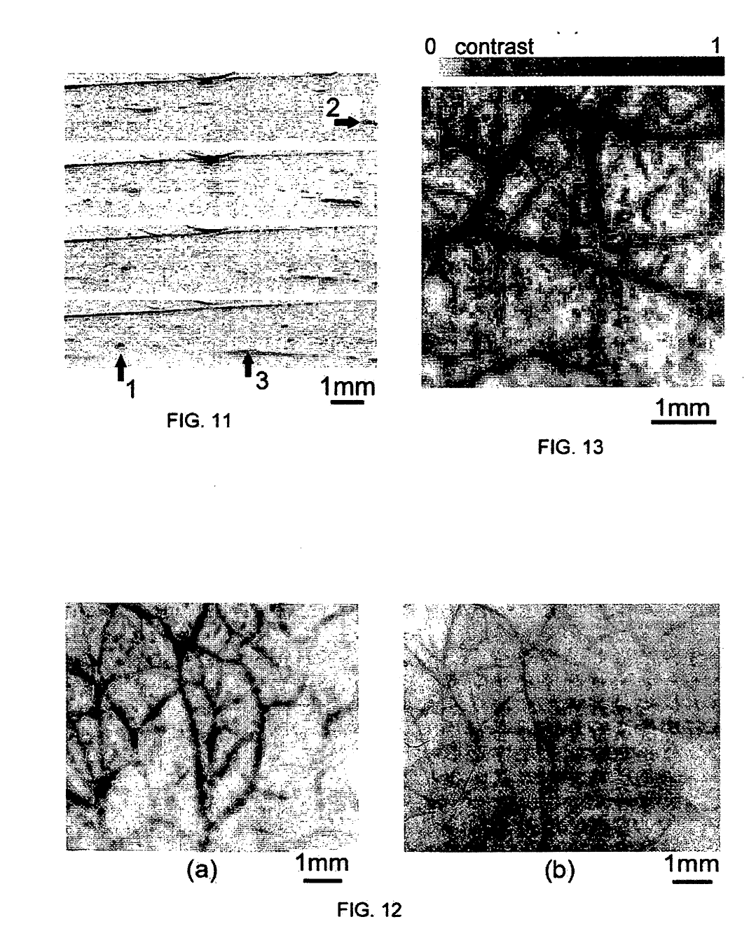

Four in situ consecutive photoacoustic B-scan images that were obtained about 0.2 mm apart laterally are shown in FIG. 11. Each image is a gray-scale plot of the peak-to-peak amplitudes of the received photoacoustic signals, where the vertical and horizontal axes represent the depth from the skin surface and the horizontal transducer position, respectively. The vertical axis was obtained by multiplying the acoustic time of arrival starting from the laser pulse with an assumed acoustic speed of about 1500 m/s. The focal plane of the transducer was located at a depth of about 1.2 mm. The animal was scanned horizontally with a step size of about 0.1 mm for 100 steps, which took about 10 seconds to complete. The slightly inclined solid line in the upper part of each B-scan delineates the skin surface. Some vessels, for example, the ones marked by 1 and 2, are nearly perpendicular to the imaging plane of the B-scans. Note that the size of vessel 1 is about 0.1 mm (one pixel) in the image, indicating that the resolution in the biological tissues is about 0.1 mm. Conversely, the vessel marked by 3 is nearly parallel with the imaging plane.

Now referring to FIG. 12, a comparison of the FIG. 12(a) in situ photoacoustic projection images taken from the epidermal side and FIG. 12(b) photographs taken from the dermal side with transmission illumination in accordance with the present invention are shown. FIG. 12(a) shows an in situ photoacoustic projection image similar to a C-scan or en face image (e.g. about 100×about 100 pixels, about 0.1 mm step size). The image is a gray-scale plot of the maximum peak-to-peak amplitudes of the received photoacoustic signals within the about 0.2 to about 2 mm depth interval from the skin surface versus the two-dimensional transducer position on the skin surface. For comparison, FIG. 12(b) shows a photograph of the inner surface of the harvested skin that was obtained using transmission illumination. Good agreement in the vascular anatomy is observed between the photoacoustic image and the photograph. Based on the photograph, the major vessels are about 100 μm in diameter, and the smaller vessels are about 30 μm in diameter.

Referring now to FIG. 13, an in vivo non-invasive photoacoustic projection image taken from the epidermal side in accordance with the present invention is shown. The photoacoustic projection image of a similar area (e.g., about 100×100 pixel, about 0.05 mm step size, about 0.5 to about 3 mm depth interval) was taken in vivo. One can see an elaborate system of blood vessels with a density of up to a few counts per mm. The signal-to-background ratio for the larger vessels is about 40 dB.

As illustrated in FIGS. 9-13, the present invention provides a dark-field confocal microscopic photoacoustic imaging technique to image biological tissues in vivo that has a lateral resolution as high as about 45 μm in tissue phantoms and a maximum imaging depth of at least about 3 mm. Further improvement of the image resolution by increasing the ultrasonic frequency is possible at the cost of the imaging depth. The photoacoustic images shown here were taken without signal averaging and, therefore, could be further improved by averaging, at the expense of data acquisition time. The current imaging speed is limited by the pulse repetition rate of the laser used. Because lasers with pulse repetition rates of up to 100 KHz are now available, real-time photoacoustic imaging, which will reduce motion artifacts, is possible and extensive signal averaging is also realistic.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All of the compositions and/or methods disclosed and claimed herein can be made and executed without undue experimentation in light of the present disclosure; While the compositions and methods of this invention have been described in terms of preferred embodiments, it will be apparent to those of skill in the art that variations can be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims

It will be understood by those of skill in the art that information and signals may be represented using any of a variety of different technologies and techniques (e.g., data, instructions, commands, information, signals, bits, symbols, and chips may be represented by voltages, currents, electromagnetic waves, magnetic fields or particles, optical fields or particles, or any combination thereof). Likewise, the various illustrative logical blocks, modules, circuits, and algorithm steps described herein may be implemented as electronic hardware, computer software, or combinations of both, depending on the application and functionality. Moreover, the various logical blocks, modules, and circuits described herein may be implemented or performed with a general purpose processor (e.g., microprocessor, conventional processor, controller, microcontroller, state machine or combination of computing devices), a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. Similarly, steps of a method or process described herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. Although preferred embodiments of the present invention have been described in detail, it will be understood by those skilled in the art that various modifications can be made therein without departing from the spirit and scope of the invention as set forth in the appended claims.

### REFERENCES

1. H. Nakajima, T. Minabe, and N. Imanishi “Three-dimensional analysis and classification of arteries in the skin and subcutaneous adipofascial tissue by computer graphics imaging,” Plastic and Reconstructive Surgery, 102, 748-760, (1998).
2. P. Carmeliet and R. K. Jain “Angiogenesis in cancer and other diseases,” Nature 407 (6801), 249-257, (2000).
3. S. J. Nelson and S. Cha “Imaging glioblastoma multiforme,” Cancer J. 9, 134-46, (2003).
4. X. Wang, Y. Pang, G. Ku, X. Xie, G. Stoica, and L. V. Wang, “Non-invasive laser-induced photoacoustic tomography for structural and functional imaging of the brain in vivo,” Nature Biotechnology 21 (7), 803-806 (2003).
5. M. Xu and L. V. Wang “Time-domain reconstruction for thermoacoustic tomography in a spherical geometry,” IEEE Trans. on Biomed. Eng., 50 (1), 1086-1099, (2002).
6. A. A. Oraevsky, V. A. Andreev, A. A. Karabutov, D. R. Fleming, Z. Gatalica, H. Singh, and R. O. Esenaliev “Laser opto-acoustic imaging of the breast: detection of cancer angiogenesis,” Proc. SPIE 3597, 352-63, (1999).
7. C. G. A. Hoelen, F. F. M. de Mul, R. Pongers, and A. Dekker “Three-dimensional photoacoustic imaging of blood vessels in tissue,” Opt. Lett. 23, 648-50, (1998).
8. R. G. M. Kolkman, E. Hondebrink, W. Steenbergen, and F. F. M. de Mul, “In vivo photoacoustic imaging of blood vessels using an extreme-narrow aperture sensor,” IEEE Journal on Selected Topics in Quantum Electronics 9, 343-346, (2003).
9. C. Guittet, F. Ossant, L. Vaillant, and M. Berson “In vivo high-frequency ultrasonic characterization of human dermis,” IEEE Trans. Biomed. Eng. v. 46, 740-746, (1999).
10. American National Standard for the Safe Use of Lasers, ANSI Standard Z136.D.H.
11. S. T. Flock, S. L. Jacques, B. C. Wilson, W. M. Star, M. J. C. van Gemert, “Optical Properties of Intralipid: A phantom medium for light propagation studies, ” Lasers in Surgery and Medicine 12, 510-519, (1992).
12. W. F. Cheong, S. A. Prahl, and A. J. Welch, “A review of the optical properties of biological tissues,” IEEE J. Quantum Electronics 26, 2166-2185 (1990).
13. W. J. Smith, “Modern Optical Engineering,” McGrew-Hill, New York, (1966), p. 318.
14. Guide for the Care and Use of Laboratory Animals, NIH Publication No. 86-23, revised. US Government Printing Office, Washington D.C., (1985).
15. Below are some example applications of photoacoustic imaging:
16. Dermatology studies [H. Nakajima, T. Minabe, and N. Imanishi “Three-dimensional analysis and classification of arteries in the skin and subcutaneous adipofascial tissue by computer graphics imaging,” Plastic and Reconstructive Surgery, 102, 748-760, (1998).]
17. Monitoring extent of tissue damage during non-invasive laser surgery [K. P Kostli, M. Frenz, H. P. Weber, G. Paltauf, H. Schmidt-Kloiber, “Pulsed Optoacoustic Tomography of Soft Tissue with a Piezoelectric Ring Sensor,” Proc. SPIE 3916, 67-74, (2000)]
18. Noninvasive detection of oral cancer in early stages [E. Savateeva, A. Karabutov, M. Matomedi, B. Bell, R. Johnigen, and A. Oraevsky, “Noninvasive detection and staging of oral cancer in vivo with confocal opto-acoustic tomography,” Proc. SPIE 3916, 55-66, (2000)]
19. Brain tumors [S. J. Nelson and S. Cha “Imaging glioblastoma multiforme,” Cancer J. 9, 134-46, (2003). X. Wang, Y. Pang, G. Ku, X. Xie, G. Stoica, and L. V. Wang, “Non-invasive laser-induced photoacoustic tomography for structural and functional imaging of the brain in vivo,” Nature Biotechnology 21 (7), 803-806 (2003).]
20. Breast tumors [A. A. Oraevsky, V. A. Andreev, A. A. Karabutov, D. R. Fleming, Z. Gatalica, H. Singh, and R. O. Esenaliev “Laser opto-acoustic imaging of the breast: detection of cancer angiogenesis,” Proc. SPIE 3597, 352-63, (1999).]
21. Additional potential applications of photoacoustic imaging are described in following literature:
22. C. G. A. Hoelen, F. F. M. de Mul, R. Pongers, and A. Dekker, “Three-dimensional photoacoustic imaging of blood vessels in tissue”, Opt. Lett. 23, 648-650 (1998).
23. R. O. Esenaliev, A. A. Karabutov, and A. A. Oraevsky, “Sensitivity of laser opto-acoustic imaging in detection of small deeply embedded tumors,” IEEE J. Sel. Top. Quant. 5, 981-988 (1999).
24. V. G. Andreev, A. A. Karabutov, and A. A. Oraevsky, “Detection of ultrawide-band ultrasound pulses in optoacoustic tomography,” IEEE T. Ultrason. Ferr. 50, 1383-1390 (2003).
25. R. A. Kruger, W. L. Kiser, D. R. Reinecke, G. A. Kruger, and K. D. Miller, “Thermoacoustic Molecular Imaging of Small Animals,” Molecular Imaging 2, 113-123 (2003).
26. X. Wang, Y. Pang, G. Ku, X. Xie, G. Stoica, and L. V. Wang, “Non-invasive laser-induced photoacoustic tomography for structural and functional imaging of the brain in vivo,” Nat. Biotech. 21, 803-806 (2003).
27. E. Savateeva, A. Karabutov, M. Matomedi, B. Bell, R. Johnigen, and A. Oraevsky, “Noninvasive detection and staging of oral cancer in vivo with confocal opto-acoustic tomography,” Proc. SPIE 3916, 55-66, (2000)
28. U.S. Pat. No. 4,058,003, “Ultrasonic electronic lens with reduced delay range”, Albert Macovski, Nov. 15, 1977.
29. U.S. Pat. No. 4,213,699, “Method of measuring low concentrations of a light absorbing component,” Moore, Jul. 22, 1980.
30. U.S. Pat. No. 4,255,971, “Thermoacoustic microscopy,” Rosencwaig, Mar. 17, 1981.
31. U.S. Pat. No. 4,267,732, “Acoustic microscope and method,” Quate, May 19, 1981.
32. U.S. Pat. No. 5,083,869, “Photoacoustic signal detecting device,” Nakata et al., Jan. 28, 1992.
33. U.S. Pat. No. 5,713,356, “Photoacoustic breast scanner,” Kruger, February 3, 1998.
34. U.S. Pat. No. 5,718,231, “Laser ultrasound probe and ablator,” Dewhurst et al., Feb. 17, 1998.
35. U.S. Pat. No. 5,840,023, “Optoacoustic imaging for medical diagnosis,” Oraevsky et al., Nov. 24, 1998.
36. U.S. Pat. No. 5,924,986, “Method and system for coherent ultrasound imaging of induced, distributed source, bulk acoustic emissions,” Chandler et al., Jul. 20, 1999.
37. U.S. Pat. No. 6,216,025, “Thermoacoustic computed tomography scanner,” Kruger, Apr. 10, 2001.
38. U.S. Pat. No. 6,498,942, “Optoacoustic monitoring of blood oxygenation,” Esenaliev et al., Dec. 24, 2002.
39. U.S. Pat. No. 6,567,688, “Methods and apparatus for scanning electromagnetically-induced thermoacoustic tomography,” Wang, May 20, 2003.

What is claimed is:

1. A method of characterizing a target within a tissue comprising the steps of:
focusing one or more laser pulses onto a surface of the tissue so as to penetrate the tissue and illuminate the target;
receiving a signal induced in the target by the one or more laser pulses using one or more ultrasonic transducers that are focused on the target; and
recording the received signal so that a characterization of the target can be obtained.

2. The method of claim 1, further comprising the step of scanning along the surface of the tissue.

3. The method of claim 1, wherein the signal comprises one or more acoustic waves, one or more pressure waves, or a combination thereof.

4. The method of claim 1, wherein the characterization of the target comprises an image of the target, a composition of the target, a structure of the target or a combination thereof.

5. The method of claim 1, further comprising the step of recording the received signal so that a characterization of the target can be obtained comprises the steps of:
generating a pressure profile from the received signal; and
recording the pressure profile so that the characterization of the target can be obtained.

6. The method of claim 1, further comprising the step of displaying an image of the target from the signal.

7. The method of claim 1, further comprising the step of recording the received acoustic or pressure waves so that a characterization of the target can be obtained comprises the steps of:
digitizing the received acoustic or pressure waves; and
transferring the digitized acoustic or pressure waves to a computer for analysis.

8. The method of claim 1, wherein the one or more laser pulses are focused with a dark field condenser.

9. The method of claim 1, wherein the one or more laser pulses selectively heat the target where optical absorption is high causing the target to expand and produce a pressure wave with a temporal profile that reflects the optical absorption of the target, the thermo-mechanical properties of the target or combinations thereof.

10. The method of claim 1, wherein the target is a small volume of the tissue.

11. The method of claim 1, wherein the target is at least a portion of an internal organ or cellular structure of a human or an animal.

12. The method of claim 1, wherein the one or more laser pulses are expanded and then converged toward a focal point of the one or more ultrasonic transducers by an optical assembly comprising one or more of lenses, one or more mirrors or combinations thereof.

13. The method of claim 1, wherein the step of receiving acoustic or pressure waves induced in the target by the one or more laser pulses using one or more ultrasonic transducers that are focused on the target is performed by scanning an annular array of ultrasonic transducers along the tissue to enhance a depth of field of an imaging system by using a synthetic aperture image reconstruction.

14. An apparatus comprising:
a focusing device that receives one or more laser pulses and focuses the one or more laser pulses onto a surface of a tissue so as to penetrate the tissue and illuminate the target; and
one or more ultrasonic transducers that are focused on the target and receive acoustic or pressure waves induced in the target by the one or more laser pulses.

15. The apparatus of claim 14, wherein the design of the apparatus allows the apparatus to be scanned along the surface of the tissue.

16. The apparatus of claim 14, wherein the focusing device comprises an optical assembly of lenses and/or mirrors that expand and then converge the one or more laser pulses toward the focal point of the one or more ultrasonic transducers.

17. The apparatus of claim 14, wherein the one or more ultrasonic transducers are positioned coaxial and confocal with the one or more laser pulses.

18. The apparatus of claim 14, wherein the focusing device comprises a dark field condensor.

19. The apparatus of claim 14, further comprising an electronic system in communication with the focusing device, the one or more ultrasonic transducers or a combination thereof, wherein the electronic system comprises an XYZ scanner, an amplifier, a digitizer, a computer, a processor, a display, a storage device or combination thereof.

20. A system comprising:
one or more pulsed lasers;
a focusing device connected to an output of the one or more pulsed lasers that receives one or more laser pulses and focuses the one or more laser pulses onto a surface of a tissue so as to penetrate a tissue and illuminate a target;
one or more ultrasonic transducers that are focused on the target and receive acoustic or pressure waves induced in the target by the one or more laser pulse; and
an electronic system that records and processes the received acoustic or pressure waves.

21. The system of claim 20, wherein the focusing device, one or more ultrasonic transducers or combinations thereof may be scanned along the surface of the tissue.

22. The system of claim 20, wherein the electronic system further comprises:
an XYZ scanner connected to the one or more ultrasonic transducers;
an amplifier and digitizer connected to the one or more ultrasonic transducers; and
a computer connected to the pulsed laser, the XYZ scanner, the amplifier and digitizer.

23. The system of claim 20, wherein the focusing device comprises an optical assembly of lenses and/or mirrors that expand and then converge the one or more laser pulses toward the focal point of the one or more ultrasonic transducers.

24. The system of claim 20, wherein the focusing device comprises a dark field condenser.
